(19) United States
(12) Patent Application Publication
(10) Pub. No.: US 2005/0010123 A1
(43) Pub. Date: Jan. 13, 2005

(54) Process for measuring QT intervals and constructing composite histograms to compare groups

(76) Inventors: Elizabeth Charuvastra, Los Angeles, CA (US); William Shell, Los Angeles, CA (US); Joan Catherine Horvath, Pasadena, CA (US)

Correspondence Address: Elizabeth Charuvastra, 3048 Nicada Drive, Los Angeles, CA, 90077, US

(21) Appl. No.: 10/617,159

(22) Filed: Jul. 9, 2003

(51) Int. Cl. A61B 5/0402

(52) U.S. Cl. 600/515

(57) ABSTRACT

A quantitative method for measuring a cardiac function interval is described as well as its application to differentiating among populations of patients. Once such populations are characterized, said method can be used as a diagnostic test for individual patients when their measured data is compared against the composite data collected by the methods herein. Beat-to-beat electrocardiographic interval data is collected over an extended period of time, such beat-to-beat data being obtained from more than one subject, the beat-to-beat interval data from each subject is then used to create a composite histogram. A series of bins representing a histogram, each of which has a value range, is defined for each subject. The collected data are organized into the bins in accordance with the value of the data and the value range of the bin, thereby creating a set of bins of each interval for each subject. A composite histogram from the set of patients is constructed by summing the data from each bin. Two composite histograms, representing two sets of observations, can then be compared using measures of central tendency, variance and outliers. This method is then applied to distinguish among populations with particular characteristics, including normal subjects persons with congenital abnormalities, and persons affected by the exposure to a pharmaceutical, toxic chemical, or other ingested or inhaled substance.

### REFERENCES CITED REFERENCED BY

U.S. Patent Documents

U.S. Pat. No. 4,417,306 November, 1983 Citron et al.

U.S. Pat. No. 5,419,338 May, 1995 Sarma et al.

U.S. Pat. No. 5,437,285 August, 1995 Verrier et al.

U.S. Pat. No. 5,560,368 October, 1996 Berger.

U.S. Pat. No. 5,560,370 October, 1996 Verrier et al.

U.S. Pat. No. 6,132,381 October, 2000 Forbes et al.

U.S. Pat. No. 6,324,423 November, 2001 Callahan and Shell

U.S. Pat. No. 6,577,894 June, 2003 Callahan and Shell

Other References

### REFERENCE LIST

1. Algra A, Tijssen J G, Roelandt J R et al. QTc prolongation measured by standard 12-lead electrocardiography is an independent risk factor for sudden death due to cardiac arrest. Circulation. 1991;83:1888-1894.
2. Algra A. Sudden death. Adv Neurol. 2003;92:221-224.
3. Bayes D L, Coumel P, Leclercq J F. Ambulatory sudden cardiac death: mechanisms of production of fatal arrhythmia on the basis of data from 157 cases. Am Heart J. 1989;117:151-159.
4. Coumel P, Leclercq J F, Leenhardt A et al. Sudden cardiac death, implanted defibrillation, and clinical electrophysiology. Pacing Clin Electrophysiol. 1991;14:893-897.
5. Coumel P, Leclercq J F, Leenhardt A. Arrhythmias as predictors of sudden death. Am Heart J. 1987;114:929-937.
6. Coumel P, Leclercq J F, Lucet V. Possible mechanisms of the arrhythmias in the long QT syndrome. Eur Heart J. 1985;6 Suppl D:115-129.
7. Fauchier L, Maison-Blanche P, Forhan A et al. Association between heart rate-corrected QT interval and coronary risk factors in 2,894 healthy subjects (the DESIR Study). Data from an Epidemiological Study on the Insulin Resistance syndrome. Am J Cardiol. 2000;86:557-9, A9.
8. Garson A, Jr., Dick M, Fournier A et al. The long QT syndrome in children. An international study of 287 patients. Circulation. 1993;87:1866-1872.
9. Leclercq J F, Coumel P, Maison-Blanche P et al. [Mechanisms determining sudden death. A cooperative study of 69 cases recorded using the Holter method]. Arch Mal Coeur Vaiss. 1986;79:1024-1033.
10. Leenhardt A, Extramiana F, Milliez P et al. [New markers for the risk of sudden death: analysis of ventricular repolarization]. Arch Mal Coeur Vaiss. 2001;94 Spec No 2:23-30.
11. Leenhardt A, Denjoy I, Maison-Blanche P et al. [Present concepts of congenital long QT syndrome]. Arch Mal Coeur Vaiss. 2000;93:17-21.
12. Lupoglazoff J M, Denjoy I, Guicheney P et al. [Congenital long QT syndrome]. Arch Pediatr. 2001;8:525-534.
13. Panidis I P, Morganroth J. Sudden death in hospitalized patients: cardiac rhythm disturbances detected by ambulatory electrocardiographic monitoring. J Am Coll Cardiol. 1983;2:798-805.
14. Schwartz P J, Wolf S. QT interval prolongation as predictor of sudden death in patients with myocardial infarction. Circulation. 1978;57:1074-1077.
15. Yi G, Guo X H, Reardon M et al. Circadian variation of the QT interval in patients with sudden cardiac death after myocardial infarction. Am J Cardiol. 1998;81:950-956.
16. Algra A, Tijssen J G, Roelandt J R et al. QTc prolongation measured by standard 12-lead electrocardiography is an independent risk factor for sudden death due to cardiac arrest. Circulation. 1991;83:1888-1894.
17. Yelamanchi V P, Molnar J, Ranade V et al. Influence of electrolyte abnormalities on interlead variability of ventricular repolarization times in 12-lead electrocardiography. Am J Ther. 2001;8:117-122.
18. Coumel P, Fayn J, Maison-Blanche P et al. Clinical relevance of assessing QT dynamicity in Holter recordings. J Electrocardiol. 1994;27 Suppl:62-66.
19. Coumel P, Leclercq J F, Naditch L et al. Evaluation of drug-induced QT interval modifications in dynamic electrocardiography: the case of bepridil. Fundam Clin Pharmacol. 1993;7:61-68.
20. Morganroth J, Hunt T, Dorr M B et al. The effect of terfenadine on the cardiac pharmacodynamics of sparfloxacin. Clin Ther. 1999;21:1514-1524.
21. Morganroth J, Hunt T, Dorr M B et al. The cardiac pharmacodynamics of therapeutic doses of sparfloxacin. Clin Ther. 1999;21:1171-1181.
22. Morganroth J, Horowitz L N. Flecainide: its proarrhythmic effect and expected changes on the surface electrocardiogram. Am J Cardiol. 1984;53:89B-94B.
23. Gallik D M, Singer I, Meissner M D et al. Hemodynamic and surface electrocardiographic effects of a new aqueous formulation of intravenous amiodarone. Am J Cardiol. 2002;90:964-968.
24. Molnar J, Weiss J S, Rosenthal J E. Does heart rate identify sudden death survivors? Assessment of heart rate, QT interval, and heart rate variability. Am J Ther. 2002;9:99-110.
25. Molnar J, Zhang F, Weiss J et al. Diurnal pattern of QTc interval: how long is prolonged? Possible relation to circadian triggers of cardiovascular events. J Am Coll Cardiol. 1996;27:76-83.
26. Molnar J, Weiss J, Zhang F et al. Evaluation of five QT correction formulas using a software-assisted method of continuous QT measurement from 24-hour Holter recordings. Am J Cardiol. 1996;78:920-926.
27. Coumel P. Diagnostic and prognostic values and limitations of Holter monitoring. Eur Heart J. 1989;10 Suppl E:19-30.
28. Coumel P, Zimmermann M. [Value of Holter monitoring in the understanding of arrhythmias]. Schweiz Rundsch Med Prax. 1986;75:1085-1089.
29. Coumel P, Leclercq J F, Slama R. [Computer analysis of ambulatory electrocardiograms. Electrophysiological value of the Holter method]. Ann Med Interne (Paris). 1986;137:632-638.
30. Coumel P, Milosevic D, Rosengarten M et al. [The theoretical and practical advantage of Holter's monitoring in sino-auricular block (author's transl)]. Ann Cardiol Angeiol (Paris). 1980;29:19-22.
31. Coumel P, Slama R. [The Holter technic: why do we use it?]. Acta Cardiol. 1980;35:169-177.
32. Leclercq J F, Maison-Blanche P, Cauchemez B et al. [Block of the atrioventricular trunk: diagnosis of the site by Holter monitoring]. Arch Mal Coeur Vaiss. 1985;78:1781-1786.
33. Leclercq J F, Coumel P. [Detection of rhythm and conduction disorders by long-term electrocardiographic recording. Holter technic]. Nouv Presse Med. 1982;11:17-19.
34. Lupoglazoff J M, Denjoy I, Berthet M et al. [T wave abnormalities on Holter monitoring of congenital long QT syndrome: phenotypic marker of a mutation of LQT2 (HERG)]. Arch Mal Coeur Vaiss. 2001;94:470-478.
35. Molnar J, Weiss J, Zhang F et al. Evaluation of five QT correction formulas using a software-assisted method of continuous QT measurement from 24-hour Holter recordings. Am J Cardiol. 1996;78:920-926.
36. Yanaga T, Adachi M, Sato Y et al. Computer analysis of Holter electrocardiogram. Fukuoka Igaku Zasshi. 1994;85:282-286.
37. Callahan T, Shell W. Quantitative Method and Apparatus For Measuring QT Intervals From Ambulatory Electrocardiographic Recodings. U.S. Pat. No. 6,324,423. 2001 November 2001.
38. Algra A, Tijssen J G, Roelandt J R et al. QTc prolongation measured by standard 12-lead electrocardiography is an independent risk factor for sudden death due to cardiac arrest. Circulation. 1991;83:1888-1894.
39. Schwartz P J, Wolf S. QT interval prolongation as predictor of sudden death in patients with myocardial infarction. Circulation. 1978;57:1074-1077.
40. Schwartz P J, Periti M, Malliani A. The long Q-T syndrome. Am Heart J. 1975;89:378-390.
41. Schwartz P J, Priori S G, Spazzolini C et al. Genotype-phenotype correlation in the long-QT syndrome: gene-specific triggers for life-threatening arrhythmias. Circulation. 2001;103:89-95.
42. Sawicki P T. Mortality in diabetic nephropathy: the importance of the QT interval. Nephrol Dial Transplant. 1996;11:1514-1515.
43. Sawicki P T, Dahne R, Bender R et al. Prolonged QT interval as a predictor of mortality in diabetic nephropathy. Diabetologia. 1996;39:77-81.
44. Yi G, Guo X H, Reardon M et al. Circadian variation of the QT interval in patients with sudden cardiac death after myocardial infarction. Am J Cardiol. 1998;81:950-956.
45. Browne K F, Zipes D P, Heger J J et al. Influence of the autonomic nervous system on the Q-T interval in man. Am J Cardiol. 1982;50:1099-1103.
46. Kautzner J, Hartikainen J E, Heald S et al. The effects of reflex parasympathetic stimulation on the QT interval and QT dispersion. Am J Cardiol. 1997;80:1229-1232.
47. Kautzner J, Camm A J. Clinical relevance of heart rate variability. Clin Cardiol. 1997;20:162-168.
48. Abadie E, Leclercq J F, Fisch A et al. [Pathogenesis of tachycardia in hyperthyroidism. Value of Holter monitoring and the use of a beta-blocker]. Presse Med. 1985;14:197-199.
49. Badilini F, Maison-Blanche P, Champomier P et al. Frequency-domain heart rate variability in 24-hour Holter recordings: role of spectral method to assess circadian patterns and pharmacological autonomic modulation. J Electrocardiol. 2000;33:147-157.
50. Carre F, Lessard Y, Coumel P et al. Spontaneous arrhythmias in various models of cardiac hypertrophy and senescence of rats. A Holter monitoring study. Cardiovasc Res. 1992;26:698-705.
51. Catuli D, Maison-Blanche P, Fayn J et al. [Analysis of frequency-dependence of ventricular repolarisation by the Holter method in young adults. Influence of the autonomic nervous system on the rate-dependence of QT]. Arch Mal Coeur Vaiss.
52. Cauchemez B, Peirano P, Samson-Dolfus D et al. [The autonomic nervous system in sudden infant death syndrome. Analysis of heart rate and sinusal variability on Holter monitoring of infants who died]. Arch Mal Coeur Vaiss. 1989;82:745-752.
53. Coumel P, Thomas O, Leenhardt A. Holter functions of the implantable cardioverter defibrillator: what is still missing? Pacing Clin Electrophysiol. 1995;18:560-568.
54. Coumel P. Diagnostic and prognostic values and limitations of Holter monitoring.
55. Coumel P, Zimmermann M. [Value of Holter monitoring in the understanding of arrhythmias]. Schweiz Rundsch Med Prax. 1986;75:1085-1089.
56. Coumel P, Milosevic D, Rosengarten M et al. [The theoretical and practical advantage of Holter's monitoring in sino-auricular block (author's transl)]. Ann Cardiol Angeiol (Paris). 1980;29:19-22.
57. Extramiana F, Tavernier R, Maison-Blanche P et al. [Ventricular repolarization and Holter monitoring. Effect of sympathetic blockage on the QT/RR ratio]. Arch Mal Coeur Vaiss. 2000;93:1277-1283.
58. Leclercq J F, Maisonblanche P, Cauchemez B et al. Respective role of sympathetic tone and of cardiac pauses in the genesis of 62 cases of ventricular fibrillation recorded during Holter monitoring. Eur Heart J. 1988;9:1276-1283.
59. Leclercq J F, Coumel P, Maison-Blanche P et al. [Mechanisms determining sudden death. A cooperative study of 69 cases recorded using the Holter method]. Arch Mal Coeur Vaiss. 1986;79:1024-1033.
60. Leclercq J F, Maison-Blanche P, Cauchemez B et al. [Block of the atrioventricular trunk: diagnosis of the site by Holter monitoring]. Arch Mal Coeur Vaiss. 1985;78:1781-1786.
61. Leclercq J F, Coumel P. [Detection of rhythm and conduction disorders by long-term electrocardiographic recording. Holter technic]. Nouv Presse Med. 1982;11:17-19.
62. Locati E H, Maison-Blanche P, Dejode P et al. Spontaneous sequences of onset of torsade de pointes in patients with acquired prolonged repolarization: quantitative analysis of Holter recordings. J Am Coll Cardiol. 1995;25:1564-1575.
63. Lupoglazoff J M, Denjoy I, Berthet M et al. [T wave abnormalities on Holter monitoring of congenital long QT syndrome: phenotypic marker of a mutation of LQT2 (HERG)]. Arch Mal Coeur Vaiss. 2001;94:470-478.
64. Lupoglazoff J M, Denjoy I, Berthet M et al. Notched T waves on Holter recordings enhance detection of patients with LQt2 (HERG) mutations. Circulation. 2001;103:1095-1101.
65. Molnar J, Weiss J S, Rosenthal J E. Does heart rate identify sudden death survivors? Assessment of heart rate, QT interval, and heart rate variability. Am J Ther. 2002;9:99-110.
66. Molnar J, Zhang F, Weiss J et al. Diurnal pattern of QTc interval: how long is prolonged? Possible relation to circadian triggers of cardiovascular events. J Am Coll Cardiol. 1996;27:76-83.
67. Molnar J, Weiss J, Zhang F et al. Evaluation of five QT correction formulas using a software-assisted method of continuous QT measurement from 24-hour Holter recordings. Am J Cardiol. 1996;78:920-926.
68. Yanaga T, Adachi M, Sato Y et al. Computer analysis of Holter electrocardiogram. Fukuoka Igaku Zasshi. 1994;85:282-286.
69. Berger R D, Kasper E K, Baughman K L et al. Beat-to-beat QT interval variability: novel evidence for repolarization lability in ischemic and nonischemic dilated cardiomyopathy. Circulation. 1997;96:1557-1565.
70. Molnar J, Zhang F, Weiss J et al. Diurnal pattern of QTc interval: how long is prolonged? Possible relation to circadian triggers of cardiovascular events. J Am Coll Cardiol. 1996;27:76-83.
71. Press W H, Teukolsky S A, Vetterling W T et al. Numerical Recipes in C: The Art of Scientific Computing. 2nd ed. Cambridge, UK: Cambridge University Press, 1994.

### FIELD OF THE INVENTION

The present invention relates to measuring cardiac function intervals.

### BACKGROUND OF THE INVENTION

It is known that alteration of the QT, QTc or RR interval on the electrocardiogram may be a marker for sudden death(¹⁻¹⁵). Measurements of the QT interval are generally taken from a 12-lead electrocardiogram where one to three heart beats are analyzed either individually or averaged(^{16;17}). The 12-lead electrocardiogram provides only point-in-time data reflecting approximately 17 seconds of time that is required to inscribe a 12-lead ECG. The QT interval duration is dynamic, however, and can vary by upwards of 100 msec in a twenty four hour period(¹⁸⁻²⁶). Thus the measurement of either a single or a few 12-lead ECGs sampled during 24 hours will miss the beat-to-beat dynamicity data that is inherent in the changes that occur. The dynamic data reflecting changes in ECG intervals is captured by longer recordings, generally 24 hours of continuous ECG data, referred to as either 24 hour ambulatory ECG (AECG) or Holter Monitoring(^{18;27-36}). Heretofore, beat-to-beat ECG data, both short and long-term recordings, has been averaged due primarily to constraints in computing power. Unfortunately, averaging minimizes the understanding of the beat-to-beat variability inherent in QT interval data. Moreover, methods to analyze large data sets of cardiac intervals have been incomplete. For example the methods for beat-to-beat binning of QT and QTc intervals described by Callahan and Shell where limited to analysis of only outliers(³⁷), calculating the % of beats that exceed a certain threshold. The disclosures by Shell and Callahan do not teach a method to analyze central tendency, variance, kurtosis or other statistical properties of the histogram as appropriate for Gausian or non-Gaussian distributions.

Increases in the QT and QTc interval measurements on a 12-lead Electrocardiogram (ECG) are associated with an increased risk of cardiac dysrhythmias and sudden cardiac death. See, for example, Algra(³⁸), Schwartz(³⁹⁻⁴¹) and Sawicki(^{42;43}). The increased QTc interval length is associated with an increased risk of sudden death from all causes. The prolongation of the QTc interval induced by pharmaceuticals has been associated with Torsade de Pointes and sudden death; the pharmaceutical induction of prolonged QTc intervals has formed the basis for removal of pharmaceuticals from the market. There is, however, no readily agreed upon method to measure the dynamic changes in the QTc interval, particularly for long term recordings of the ECG.

While the resting 12-lead electrocardiogram may provide important spatial information regarding the status of ventricular repolarization, the use of a single 12-lead ECG measured randomly in time may disregard potentially important prognostic data regarding the dynamicity, temporal relationships, and circadian rhythms of the QT interval.

It is known that the QT interval may undergo significant changes over both the short and long term due to circadian rhythms. See, for example, Yi, et al(⁴⁴) who teach the association between circadian rhythm and sudden death associated with acute myocardial infarction. See also, for example, Callahan and Shell who describe a method to assess circadian changes in the QT interval.

It is known that the QTc interval may undergo significant changes over both the shorter and longer term due to autonomic control. See, for example, Cappatto et al, Browne et al(⁴⁵), and Kautzner, et al(^{46;47}), demonstrated the relationship between sympathetic and vagal tone on the QT and QTc interval.

Thus, a single 12-lead ECG taken at a given point in time may provide misleading and inaccurate cardiac risk data. Therefore, analysis of the QT interval for an entire 24-hour period, reflecting circadian and autonomic changes, may provide additional information regarding the risk of sudden death not available on the single, random 12-lead ECG.

It is now possible to measure the QT interval on 24-hour Holter (AECG) recordings(^{18;29;31;48-64}) These measurements have generally been reported as averages over short time periods, typically between about 15 seconds and about five minutes, for example Molnar et al(⁶⁵⁻⁶⁷) or Yanaga, et al(⁶⁸). The use of averaged QT measurements may obscure significant short-term variations in the QT intervals. Conversely, beat-to-beat measurements retain the natural variability data that may be important for calculating a patient's risk of dysrhythmia and sudden death.

More recently beat-to-beat QT interval measurements have been used but methods to analyze the beat-to-beat changes have been incomplete.

Although beat-to-beat variability of the QT interval has been described by Berger and others (⁶⁹), little is known regarding normal ranges in variability and measures of the QT interval over a 24-hour period using beat-to-beat measurements.

Molnar and colleagues published a study that gives some indication of the dynamic range of the QT intervals using five minute averages and not beat-to-beat measurements(⁷⁰). They reported a mean maximum QTc interval of 495 ms for normal subjects using 24-hour ambulatory monitoring. They also showed a mean intra-subject change of 95 ms. Molnar further reported six normal female subjects as having a maximum mean QTc interval measurement of more than 500 ms. These mean maximum measurements were taken over a five-minute period.

The use of average QTc measurements obscures the dynamicity of individual beats. Measurements of central tendency, skewness and shape of histograms have not been used extensively to describe the relationship of QT and QTc measurements in histograms representing beat-to-beat QT, QTc or RR intervals. These measurements may be important to give an overall picture of the status of the subject.

It is an objective of the present invention, in a preferred embodiment, to enable the assessment of the QT and QTc intervals and other cardiac function intervals on a beat-to-beat basis, providing a composite histogram of the individual beats with QT and QTc intervals.

It is another objective of the present invention, in a preferred embodiment, to enable the measurement and assessment of the QT and QTc intervals and other cardiac function intervals over an extended period of time, including not only periods of time greater than about one minute but also periods of time lasting at least 24 hours and even longer, in some cases.

### SUMMARY OF THE INVENTION

In accordance with the present invention, in a preferred embodiment, this and other objectives are achieved by providing a method for analyzing beat-to-beat QT intervals from high-resolution Ambulatory Electrocardiographic monitoring (AECG) to detect the frequency distribution in a continuous AECG recording. Beat-to-beat QT and RR intervals may be measured to calculate beat-to-beat QTc. In a preferred embodiment, a composite of the entire frequency distribution of QT and QTc intervals taken from a set of observations with a common characteristic may be examined. Moreover, a composite of one characteristic may be statistically compared to a composite with other characteristics, including statistical methods that do not assume a normal distribution of the histogram.

The present invention, in a preferred embodiment, provides a method to analyze beat-to-beat QT data, stratify the data according to a time-series bin-array, and create a composite of multiple histograms. This method and apparatus may be applicable to a wide variety of different subjects including, for example, normal subjects, subjects with the Inherited Long QT syndrome (ILQTS), and subjects exposed to drug titration. The statistical characteristics of a normal subject group can be compared to either a second group, or to individuals who have taken a drug, have potential congenital heart disease, have been exposed to an environmental toxin, or have a disease which could cause prolongation of the QTc interval such as diabetes mellitus.

Further objects, advantages and other features of the present invention will be apparent to those skilled in the art upon reading the disclosure set forth herein.

### DESCRIPTION OF THE ILLUSTRATIONS

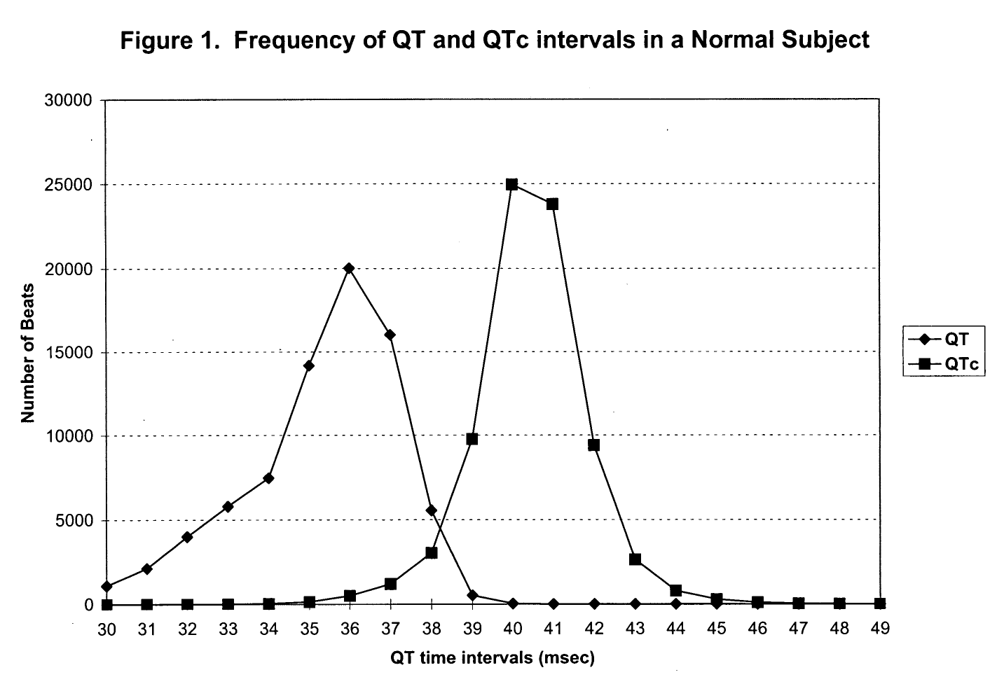

FIG. 1. Frequency of QT and QTc intervals in a Normal Subject

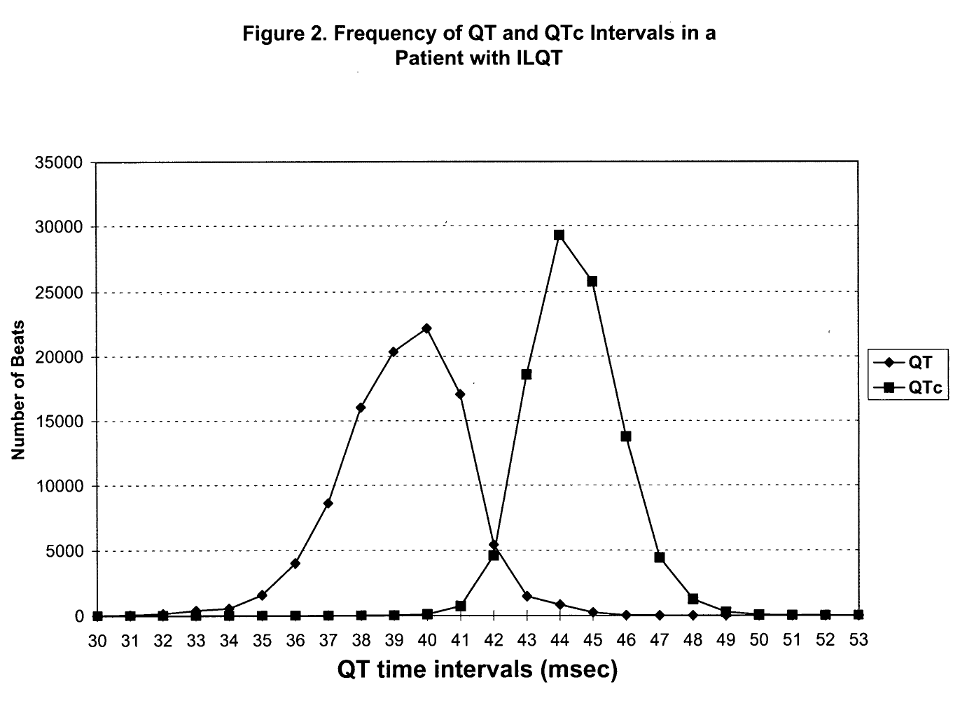

FIG. 2. Frequency of QT and QTc intervals in a Patient with ILQT

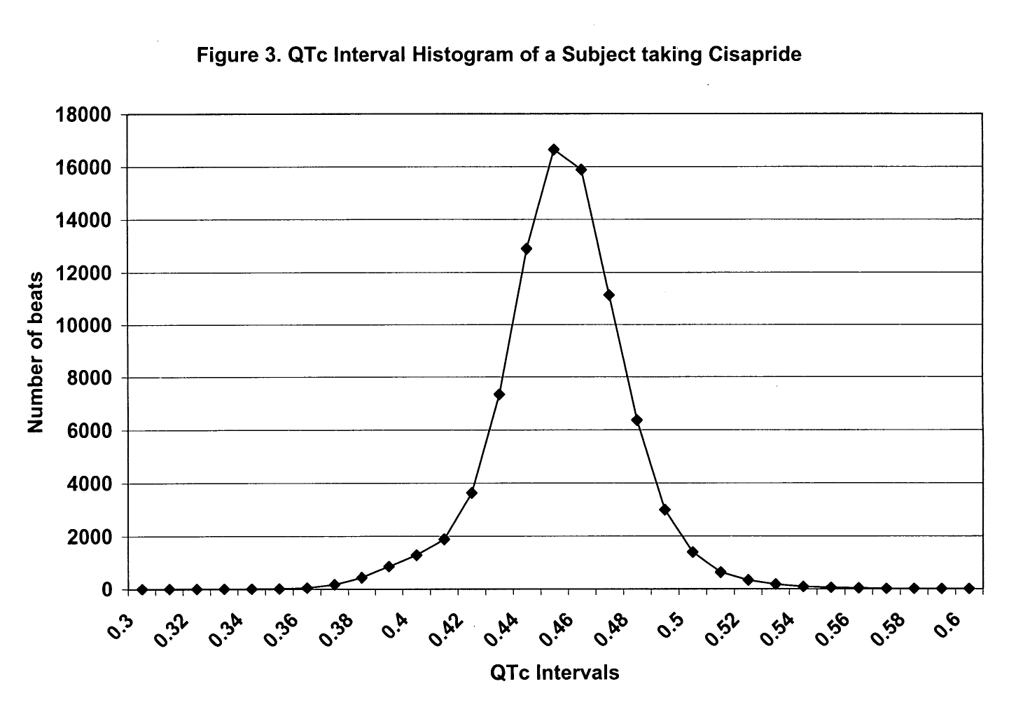

FIG. 3. QTc Interval Histogram of a Subject taking Cisapride

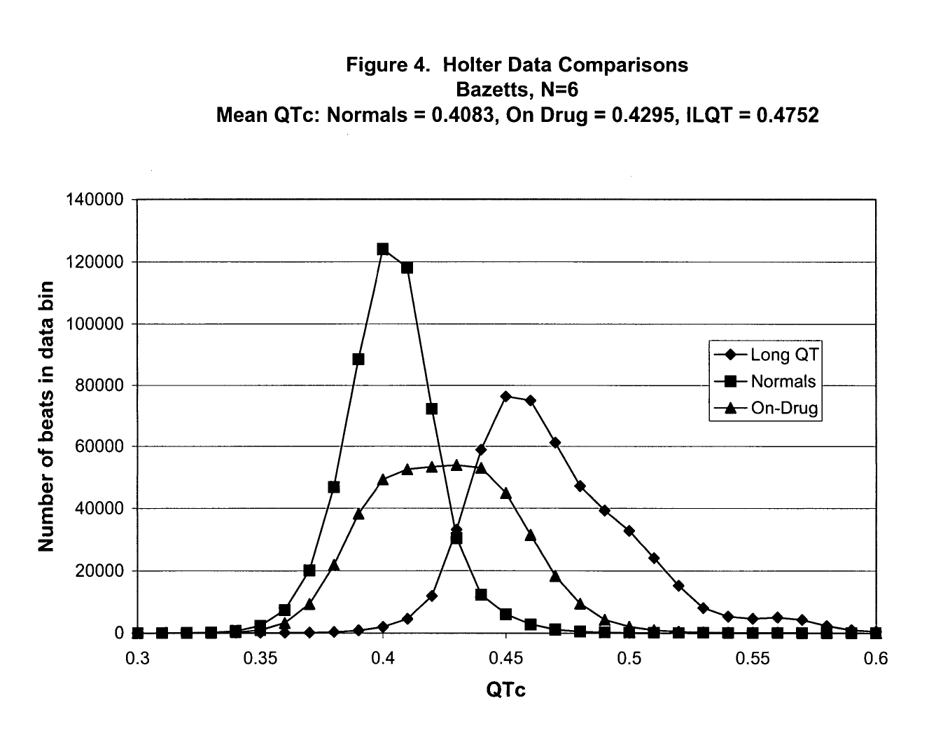

FIG. 4. Holter Data Comparisons of composite curves from normal subjects, subjects on cisapride and subjects with Inherited Long QT Syndrome (ILQT)

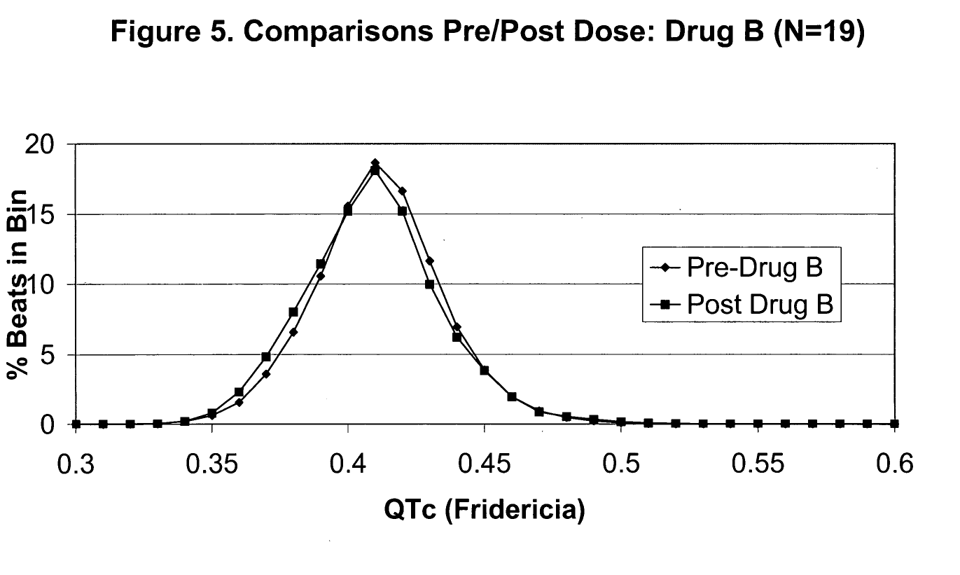

FIG. 5. Comparisons Pre/Post Dose of Drug using composite curves (N=19).

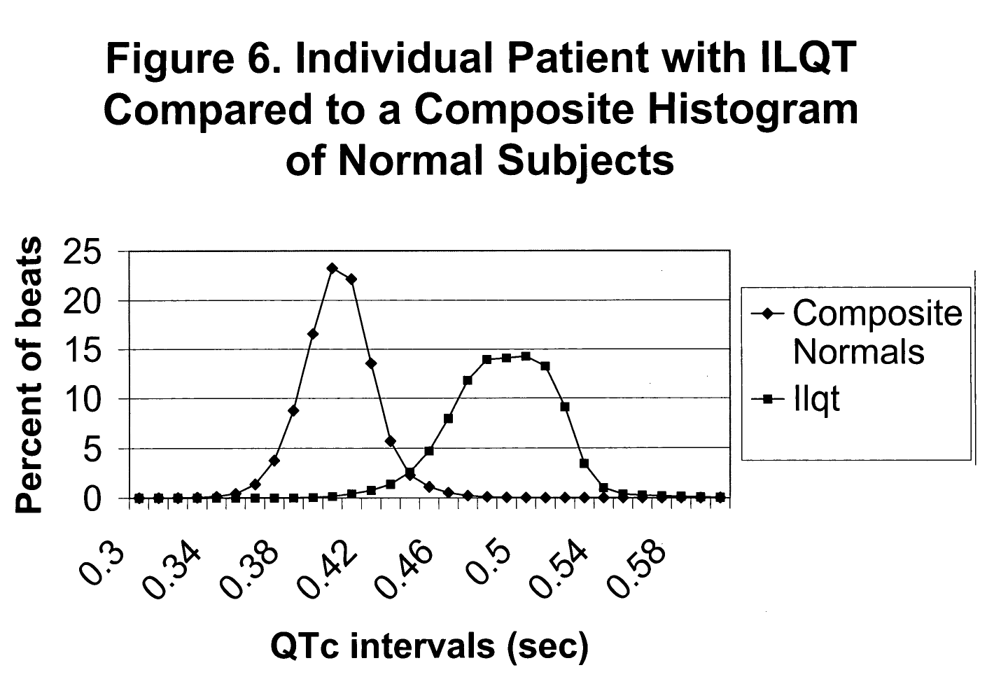

FIG. 6. Individual Patient with ILQT Compared to a Composite Histogram of Normal Subjects

### DETAILED DESCRIPTION OF THE PREFERRED EMBODIMENTS

The following detailed description is of the best presently contemplated mode of carrying out the invention. This description is not to be taken in a limiting sense, but is made merely for the purpose of illustrating the general principles of the invention. The scope of the invention is best defined by the appended claims.

In a preferred embodiment, standard 24-hour AECG recordings may be obtained using any commercially available Holter cassette tape recording device. An example of this type of device is a Reynolds Medical Tracker II (Reynolds Medical, Hertford UK) recorder. Also in the preferred embodiment, standard 24-hour AECG recordings can be obtained using commercially available digital Holter recorders that have a sufficient sample rate to allow detection and measurement of the cardiac intervals. For QT interval analysis, the sample rate can be between 128 and 2000 samples per seconds. The preferred embodiment is the use of a sample rate of at least of at least 1000 samples per second. These digital recorders must also be compatible with a Holter playback system that can produce beat-to beat interval measurements. An example of this type of device is the Reynolds Medical LifeCard CF recorder (Reynolds Medical, Hertford, UK). An example of the compatible Holter playback system is the Reynolds Medical Pathfinder 700 series. (Reynolds Medical, Hertford, UK). These recorders and playback systems are commercially available and need not be modified.

Analog signals from the Holter cassette recordings may be digitized at 12-bit or higher resolution using a Holter playback system that has the ability to perform interval measurements. The Reynolds Medical Pathfinder 700 series Holter analyzer is an example of this type of equipment (Reynolds Medical, Hertford, UK).

Using the digitized file of the electrocardiogram, a QT interval analysis may be accomplished in the following manner: The onset of a Q-wave (Qb) may be defined and a cursor may be placed at this point. The end of a T-wave (Te) may be defined and a second cursor may be placed at this point. The data from the digital file may then be replayed at 60-times normal time, while the cursors on the Qb and Te points may be monitored for stability. If either cursor wavers from the Qb or Te points, the cursors may be replaced and the affected portion of the data may be reanalyzed. The QT interval may be defined as the time difference between the time points at Qb and Te. The QT intervals may be measured for the entire AECG recording on a beat-to-beat basis. Other analysis systems that display digital data may be used.

The peak of an R-wave may be detected and a third cursor may be placed (Rp). Accordingly, each QT interval may be matched with the preceding R--R interval. For a 24-hour recording, this may result in approximately 100,000 beats for which a QT interval and an R--R interval may be defined. The data may then be output to a high-speed computer for post-analysis processing.

In the examples described herein use was made of AECG recordings from normal volunteers, subjects treated with placebo and subjects on-treatment in a drug treatment study, and recordings from subjects with inherited Long QT Syndrome (ILQT). These recordings help to demonstrate the potential effectiveness creating composite curves in accordance with the present invention.

In the examples described herein QTc was calculated by removing a time-series of the QT and preceding R--R intervals to a high-speed computer with both a fast processor and adequate disk storage space. For each QT interval, a QTc may be calculated using a variety of correction factors for the QT interval including Bazett's correction formula, Fridericia correction formula and linear correction formula.

The QT and QTc intervals may be individually placed in the bins according to their measurement as described in Shell and Callahan. In a preferred embodiment the composite curves are constructed by software programs that generate a time series of approximately 100,000 data points long of RR/QT/QTc triplets for each patient. Then the QTc data for each patient is binned in a histogram for that patient, finally, software is used to merge many patients'data into a composite data set (a“population”) and to take means and standard deviations of this population (assuming normalcy of the data). Finally, more the data thus aggregated into two or more populations can then be compared, again using a combination of software and procedures as described in Press et al(⁷¹), against each other to check for statistical difference between these two or more populations.

These aggregated population curves can then be used as a template for comparison against an single patient's binned histogram to determine what population (e.g. normal, inherited disorder, or drug-induced damaged) this particular patient belongs. The current embodiment assumes normal distributions, but this is not intrinsic to the method and more sophisticated distribution-distinguishing numerical analysis and statistics is declared here as well. The numeric procedures used are commonly described in Press, et al.

### EXAMPLE1—NORMAL SUBJECT

FIG. 1

In this example, the 24 hour ambulatory ECG from a normal subject was analyzed. The 24-hour ECG was digitized. The QT and RR interval was determined for each beat using the Reynolds's analyzer. All extra beats were eliminated. All beats with prolonged QT intervals were inspected and artifact was eliminated. The QT and RR files were then used to construct a Histogram of QT and QTc intervals. The histogram was constructed with 10 msec intervals. The histogram of QTc intervals is depicted in FIG. 1. The normal subject had a mean QT interval measurement of 358 msecs with a standard deviation of 37 msecs. the mean QTc measurement was 409 msecs with a standard deviation of 13 msecs.

### EXAMPLE 2

FIG. 2

PROLONGED QTc Intervals in Inherited Long QT Syndrome.

The Inherited Long QT Syndrome is a genetic defect of the heart's ion channels. The patients with Inherited Long QT Syndrome are known to have intermittent prolonged QTc intervals. Often, however, many of the heart beats of patients with inherited long QT syndrome are within the normal range and the identification of these patients cannot be made from a single conventional 12 lead ECG. Since these patients, often children, die suddenly, failure to detect the presence of the abnormal gene can lead to sudden death of the infant, child or young adult, an unnecessary death since treatment is available to prevent such sudden death. In this example., a child with a known gene defect underwent 24 hour ambulatory monitoring. The ECG was digitized and the QT and RR intervals defined. The QT and QTc histograms are depicted in FIG. 2. The mean QTc was 450 msec with a standard deviation of 20 msec .

### EXAMPLE 3

FIG. 3

Patients with Drug Induced Long QT Interval.

Many drugs can prolong the QTc interval and the drug induced prolonged QTc interval is associated with an increased incidence of sudden death. Many drugs have been removed from the market because they prolong the QTc interval. Cisapride is a drug that can prolong the QTc interval. In FIG. 3, the QTc interval histogram is depicted in a patient taking cisapride. The mean QTc interval was 440 msec.

### EXAMPLE 4

FIG. 4

Comparison of the QTc Interval Using Composite Curves in Normal Subjects to those with Inherited Long QT Syndrome and Drug Therapy

In FIG. 4, a composite curve was generated from six normal subjects and a composite curve was generated from six subjects with known Inherited Long QT Syndrome. The mean QTc from the normal subjects was 409 msec+/−20 msec while the mean QTc from the patients with Inherited Long QT Syndrome was 475 msec+/−35 msec and the drug therapy was 430 msec+/−0.40 msec. The kurtosis for the normals was 525 while the skewness was 767. The kurtosis for the patients with the gene defect was 1.24 while the skewness was 0.203.

### EXAMPLE 5

FIG. 5

Comparison of the QTc Interval Using Composite Curves in a Subject Before and After Drug Intervention

Since one of the important uses of this methodology is to compare the QTc interval before and after the use of a pharmaceutical that could prolong the QTc interval, we compared a group of patients before and after the administration of a pharmaceutical. The 19 patients had 24 hour ambulatory ECG monitoring before and after the administration of drug. The composite curves before and after the administration of drug are depicted in FIG. 5. The mode before was 391 msec and after was 392 msec. Using a paired t-test the p-value was 0.98. The total number of beats analyzed before treatment was 8.5 million beats and was 8.7 million beats after treatment. The use of such composite curves generates large data sets that allow determination of difference/no difference in treatment sets with a high degree of statistical reliability. The conventional method to define differences would have analyzed between 50 and 3000 beats taken from 12 to 128 patients on resting 12-lead ECG.

We then compared the means in these two groups using a standard T-test and analysis of variance. The p-value for the difference was less than 0.000008. The creation of the composite curves allows definitive differentiation of the three groups.

### EXAMPLE 6

FIG. 6

Comparison of a Single Individual to a Composite Set of Data

Frequently, one is confronted with the problem of defining if a set of QTc data is derived from a set of normal data. In this example, a single individual with Inherited Long QT Syndrome was compared to a set of normal subjects (FIG. 6). In this example, the mean for the normal set was 408 msec while the mean for the patient with IQLT was 501 msec. Then the ILQT patient's histogram was compared to the normal set by use of either analysis of variance or Student t-test, the p-value was less than 0.00000001 indicating that the likelihood of the patient's histogram was sampled from the same population set as the normal of less than one in a million. This degree of statistical reliability would form the basis of a diagnostic test for patients with suspected ILQT. In this case the composite curve was comprised of 533,354 beats compared to 94,996 beats for the individual patient histogram. If the patient had a 12-lead ECG, there would have been fewer than 20 beats available to compare the QTc interval to a mean normal that did not account for the beat-to-beat dynamicity of the QTc interval. This example shows how the composite curve invention can be used as a diagnostic test.

In a preferred embodiment, the present invention represents a new method for quantifying the QT and QTc interval measurements over a period of time. The invention allows a quantitative comparison of two or more sets of QT or QTc intervals. For example, the invention allows comparison of a group of patients before and after a drug. The method described allows application of a variety of statistical methods to define whether two or more sets of intervals are different from one another.

In a preferred embodiment, the method and apparatus may make use of high-speed computer processors, and large capacity data-storage media. In a preferred embodiment a 1 GHz Pentium IV processor with an 80-gigabyte hard drive may be used to analyze and store the large data files. Several custom-built software programs are used to generate a time series of approximately 100,000 data points of RR/QT/QTc measurements for each patient. Then the QTc data for each patient is binned in a histogram for that particular patient, finally, a combination of software and procedures are used to merge many patients'data into a composite data set (a“population”) and to take means and standard deviations of this population (assuming normalcy of the data). Finally, the data thus aggregated into two or more populations can then be compared, again using a combination of custom software and procedures, against each other to check for statistical difference between these two or more populations.

These aggregated population curves can then be used as a template for comparison against an single patient's binned histogram to determine what population (e.g. normal, inherited disorder, or drug-induced damaged) this particular patient belongs. The current embodiment assumes normal distributions, but this is not intrinsic to the method and more sophisticated distribution distinguishing numerical analysis and statistics is declared here as well.

Composite QTc histogram measurements in accordance with the present invention allows for a quantitative assessment of the number of specified intervals, such as QT and QTc in a 24-hour AECG recording.

The present invention, in a preferred embodiment, is directed to a method for the quantification of beat-to-beat QT and QTc interval measurements from ambulatory electrocardiographic recordings.

A QT binning technique in accordance with the present invention may be used to provide information about the effects of a pharmaceutical. For instance, in the example illustrated in FIG. 5, patients had two separate 24 hour AECG recordings. The first was a base line ECG recording. Then a pharmaceutical agent was was given to the patients in random order and the patients were monitored. Using a binning method and construction of composite curves in accordance with the present invention, an increase in the QT interval could be demonstrated better than by simply averaging or measuring a QT interval.

Although the preferred embodiment of the present invention has been described herein with respect to measurement and analysis of the QT interval, it will be recognized that a method in accordance with the present invention may also be useful in the measurement and analysis of a wide variety of other ECG and related biologically significant intervals

In a preferred embodiment, the method takes discreet measurements and discreet intervals and places them into a time series bin or an amplitude series bin. For example, all of the RR intervals in a sample could be selected and coded according to their length and then placed into bins. Each bin could be characterized by a frequency. The same analysis could be performed using any interval on the electrocardiogram.

The presently disclosed embodiments are to be considered in all respects as illustrative and not restrictive, the scope of the invention being indicated by the appended claims, rather than the foregoing description, and all changes which come within the meaning and range of equivalency of the claims are therefore intended to be embraced therein.

1. A quantitative method of measuring a cardiac function interval, the method comprising:
collecting from a continuous recording of a cardiac interval taken from a single individual obtained over an extended period of time, beat-to-beat data representative of a cardiac interval, each beat-to-beat data having a value,
defining a plurality of bins, each one of the plurality of bins having a defined value range,
organizing each of the collected data into one of the plurality of bins in accordance with the value of the data and the value range of the bin to create a histogram,
constructing a composite histogram by summing the contents of each bin from a set of individual histograms derived from a group of recordings taken from several individuals with common characteristics, and
performing a statistical analysis on the combined histogram to define the statistical characteristics of the group, where such analysis can, but does not necessarily require Gaussian (“normal”) distribution of the data in said group.

2. The method of claim 1 wherein the step of summing of each individual bin comprises calculating a composite set of data.

3. The method of claim 1 wherein the representative interval comprises a time measurement.

4. The method of claim 1 wherein the interval comprises an amplitude measurement.

5. The method of claim 1 wherein the step of collecting data comprises obtaining an ambulatory electrocardiographic monitoring recording.

6. The method of claim 1 wherein the cardiac function interval comprises at least one of a QT interval, a QTc interval, a PR interval, an RR interval, an ST interval, a QRS duration, a JT interval, an interval between QTA apex and QTE end of T-wave, and an interval between P beginning and P end.

7. A quantitative method of measuring a cardiac function interval, the method comprising:
collecting from a continuous recording of a cardiac interval taken from a single individual obtained over an extended period of time, beat-to-beat data representative of a cardiac interval, each beat-to-beat data having a value,
defining a plurality of bins, each one of the plurality of bins having a defined value range,
organizing each of the collected data into one of the plurality of bins in accordance with the value of the data and the value range of the bin to create a histogram,
constructing a composite histogram by summing the contents of each bin from a set of individual histograms derived from a group of recordings taken from several individuals with a common characteristics, and
performing a statistical analysis comparing one composite histogram taken from a group of subjects having one common characteristic to a second or more composite histograms taken from a second or more group of subjects having a second or more characteristic to define whether the group or groups have been sampled from the same population.

8. The method of claim 7 wherein the step of summing of each individual bin comprises calculating a composite set of data.

9. The method of claim 7 wherein the representative interval comprises a time measurement.

10. The method of claim 7 wherein the representative interval comprises an amplitude measurement

11. The method of claim 7 wherein the means for collecting data comprises ambulatory electrocardiographic monitor.

12. The method of claim 1 wherein the cardiac function interval comprises at least one of a QT interval, a QTc interval, a PR interval, an RR interval, an ST interval, a QRS duration, a JT interval, an interval between QTA apex and QTE end of T-wave, and an interval between P beginning and P end.

13. A method of measuring an effect of a pharmaceutical or other therapeutic agent on a subject, comprising:
providing a pharmaceutical or other therapeutic agent to the subject,
collecting, over an extended period of time, beat-to-beat data representative of a cardiac interval of the subject, each beat-to-beat data having a value,
defining a plurality of bins, each one of the plurality of bins having a defined value range,
organizing each of the collected data into one of the plurality of bins in accordance with the value of the data and the value range of the bin, and
calculating a sum of data in each bin based upon the quantity of data in each bin to create a composite histogram, and.
statistically analyzing the composite histogram after exposure to the pharmaceutical or other therapeutic agent, baseline or placebo.

14. A quantitative method of measuring a cardiac function interval, the method comprising:
collecting, over an extended period of time, beat-to-beat data representative of a cardiac interval, each beat-to-beat data having a value,
stratifying the collected data, based upon the value of the collected data, in accordance with a plurality of defined bins, each one of the plurality of bins having a defined value range, and
creating a composite histogram to allow statistical analysis of the histogram.

15. A quantitative method of measuring a cardiac function interval, the method comprising:
collecting, over an extended period of time, beat-to-beat data representative of a cardiac interval, each beat-to-beat data having a value,
stratifying the collected data, based upon the value of the collected data, in accordance with a plurality of defined bins, each one of the plurality of bins having a defined value range, and
creating a composite histogram to allow statistical analysis of the histogram, and comparing an individual patient histogram to a composite curve.

16. A method as in claim 15 where the composite curve is derived from a set of normal subjects and the individual histogram is tested to assess the probability that the individual histogram falls within the set of normal subjects.

17. A method as in claim 15 where the composite curve is derived from a set of placebo treated subjects and the individual histogram is tested to assess the probability that the individual histogram falls within the set of placebo subjects.

18. A method as in claim 15 where the comparison of the individual histogram to the composite curve is used as a diagnostic test to determine the probability that the individual is derived from the set utilized to construct the composite curve.

19. A method as in claim 15 where the composite curve is derived from a set of either normal subjects, placebo treated subjects or subjects with other baseline characteristics and the individual histogram is derived from either a potential normal subject or a subject with disease.
